(19) United States
(12) Patent Application Publication
(10) Pub. No.: US 2021/0322594 A1
(43) Pub. Date: Oct. 21, 2021

(54) METHODS AND SYSTEMS FOR DISINFECTION USING UVC LIGHT

(71) Applicants: Khalid Ahmad, Durham, NC (US); Petrus Paulus Jonker, Delft (NL); Robertus Gerardus Theodorus Van Hamersveld, San Ramon, CA (US)

(72) Inventors: Khalid Ahmad, Durham, NC (US); Petrus Paulus Jonker, Delft (NL); Robertus Gerardus Theodorus Van Hamersveld, San Ramon, CA (US)

(21) Appl. No.: 17/217,761

(22) Filed: Mar. 30, 2021

Related U.S. Application Data
(60) Provisional application No. 63/013,377, filed on Apr. 21, 2020.
(60) Provisional application No. 63/064,657, filed on Aug. 12, 2020.
(60) Provisional application No. 63/081,721, filed on Sep. 22, 2020.

(51) Int. Cl. A61L 2/10; A61L 2/24; A61L 2/26

(52) U.S. Cl. CPC A61L 2/10; A61L 2/24; A61L 2202/25; A61L 2202/14; A61L 2202/16; A61L 2/26

(57) ABSTRACT

Provided herein are embodiments of systems and methods for disinfecting a passenger cabin using UVC light. One of the systems includes: a housing; one or more UVC light sources configured to emit light having wavelength between 200 to 280 nm, wherein the one or more UVC light sources are mounted on the housing; a sensor configured to determine whether the vehicle is empty of occupants; and a controller configured to control the one or more UVC lights based on the sensor determination of whether the vehicle is empty.

### CROSS-REFERENCE TO RELATED APPLICATIONS

This application claims priority to U.S. Provisional Application No. 63/013,377 entitled “Systems and Methods for Automotive Disinfection Utilizing UV-C Light”, filed Apr. 21, 2020; to U.S. Provisional Application No. 63/064,657, entitled “UV-C Light Application for Disinfection In The Transportation Industry”, filed Aug. 12, 2020; and to U.S. Provisional Application No. 63/081,721, entitled “UV-C Application for Sun Visor, filed Sep. 22, 2020, these applications are incorporated herein by reference in their entireties for all purposes.

### TECHNICAL FIELD

The disclosure relates generally to the field of disinfection systems, specifically and not by way of limitation, some embodiments are related to disinfection system of personal spaces.

### BACKGROUND

UV light is a form of electro-magnetic radiation with wavelengths shorter than visible light. UV light can be separated into various ranges. For example, short-wavelength UV (UVC) is between about 200 and 300 nm and is considered “germicidal UV”. According to several studies, the UV-C light or UVC disarms the RNA in viruses like SARS-CoV2 and it harms their lipid and protein body. This means that the virus is still there, but it cannot procreate. Likewise, for DNA viruses, bacteria, and fungi; UVC brings their DNA in disarray so they cannot procreate and grow anymore. The reason behind this is that UVC light is not naturally found on earth, as it is filtered out in the earth's atmosphere, and hence during evolution there was no need for life on earth to invent shielding against UVC.

As UVC light is not naturally found on earth it is also dangerous for human skin and especially for eyes. Short exposure immediately gives a reaction that can best be described as a sunburn reaction and for the eye as snow-blindness. On the longer run it might give rise to skin cancer as UVC might also bring the DNA of the skin cells in disarray. Living creatures such as humans and animals must not be exposed to UVC light. So when disinfecting, no humans or animals must be around in the immediate vicinity, the distance depending on the irradiation power of the UVC source. Glass and other clairvoyant materials such as Perspex block UVC.

It is known that viruses stay in aerosols for up to three hours, after which they usually end up on the floor or walls or surfaces such as tables, chairs, etc. They can survive on copper for 4 hours, on cardboard for 24 hours, on textile for 48 hours and on stainless steel and plastic for up to 72 hours. Fortunately, man can create UVC light to disinfect surfaces and air from viruses, bacteria and fungi. Accordingly, what is needed is a UVC disinfection system that can be easily used to disinfect high-use spaces, surfaces and areas.

A typical UVC energy value for distorting the ability of viruses like SARS-CoV2 on a surface to procreate is >50 nJ/cm². This means that given the irradiation power of the UVC source, the distance of the source to the surface, as well as the angles between source and surface, a certain time is required to disinfect a space. Feasible times are minutes to hours, depending on the required use of the space to be disinfected and the complexity of its geometry. Indirect irradiation, i.e. shadowed areas may need more time to reach the 50 nJ/cm². Furthermore, the necessary irradiation power depends on the available disinfection time; i.e. overnight disinfection needs less powerful UVC sources than disinfection within minutes.

### SUMMARY

Provided herein are embodiments of systems and methods for disinfecting a person's confined personal space using UVC light, whereas the system disables disinfection when humans and/or animals are detected in that space by the system. One of the embodiments includes the seat—with space around the seat within hands reach—of a vehicle, such as the driver seat and passenger seats of cars, trucks, buses, boats or aircrafts. Other embodiments include cabins such as toilets, shower cabins, or elevators.

One of the embodiment of the system includes: a housing; with one or more UVC light sources configured to emit light having wavelength between 200 to 280 nm, wherein the one or more UVC light sources are mounted in the housing; a sensor configured to determine whether the confined personal space to disinfect is empty of occupants; and a controller configured to control the one or more UVC lights based on the sensor determination of whether the space is empty.

One of the methods for disinfecting a cabin of a vehicle includes: determining, using one or more sensors to determine whether the confined personal space is empty of occupants and turning on a UVC light source to disinfect the confined personal space based on whether this space is empty of occupants.

One of the embodiment of the system includes: a housing; with one or more UVC light sources configured to emit light having wavelength between 200 to 280 nm, wherein the one or more UVC light sources are mounted in the housing; a sensor configured to determine whether the confined personal space to disinfect is empty of occupants; a controller configured to control the one or more UVC lights based on the sensor determination of whether the space is empty and a wire-full and/or wire-less network to connect remote sensors, such as door-sensors, remote monitoring and control systems, such as apps on smartphones, and to synchronize disinfection operations between multiple of such disinfection systems for confined personal spaces, such as the simultaneous disinfection of some seats of a car, bus, boat or airplane, etc. or the simultaneous disinfection of some toilets, shower cabins, etc. in a gym, restaurant, etc.

One of the methods for synchronized disinfecting of confined personal spaces includes: determining, using one or more sensors to determine whether the confined personal spaces of a predetermined group are empty of occupants and turning on the UVC light sources of the systems of the group to disinfect the confined personal spaces based on whether this group of spaces is empty of occupants. By way of example, all seats in a car, only the front seats, all cars in a garage, all back seats of an airplane, etc.

Other features and advantages of the present invention will be or will become apparent to one with skill in the art upon examination of the following figures and detailed description, which illustrate, by way of examples, the principles of the present invention.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 illustrates a visor shaped housing with a UVC disinfection system in accordance with some embodiments of the present disclosure.

FIG. 2 illustrates a circuit diagram of the UVC disinfection system in accordance with some embodiments of the present disclosure.

FIG. 3 illustrates an example environment where one or more UVC disinfection systems can be implemented in accordance with some embodiments of the present disclosure

FIGS. 4 and 5 illustrate side views of environments in which such a UVC disinfection system can be implemented in accordance with some embodiments of the present disclosure.

FIG. 6 illustrates a clip-on to a visor with a UVC disinfection system in accordance with some embodiments of the present disclosure.

FIGS. 7 and 8 are flow charts of disinfecting processes in accordance with some embodiments of the present disclosure.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

### Overview

In order to disarm 99.99% of viruses like SARS-CoV2 an energy of 50 mJ/cm2 is sufficient. Like normal light, UVC cast shadows, this means that at shadowy places this energy must be thrown on the surface by longer illumination. Alternatively, the UVC light source can rotate and/or move in order to better expose different areas of an environment (e.g., the ally of a bus or airplane). UVC is dangerous for human skin and especially for eyes, short exposure immediately gives a reaction that can best be described as a sunburn reaction and for the eye as snow-blindness. On the longer run it might give rise to skin cancer as UVC brings also DNA of the skin cells in disarray. Humans and animals must not be exposed to UVC light even as it is still at long distances. It should be noted that glass (i.e., cars windows and windshield) blocks UVC light. Accordingly it is safe to stand behind a pane of glass or a closed car window. Alternatively a Perspex shield between front seats and back seats can be used by a taxi-driver to disinfect the back-seats while driving to the next customer.

Disclosed herein is a UVC disinfection system that can be mounted on various places inside of a vehicle (e.g., cars, trucks, commercial vehicles, emergency vehicles, planes, boats, trains, buses) such as, but not limited to the visor, the ceiling of the passenger cabin, behind a seat headrest, front/back pillars of an automobile, etc. For example, the UVC disinfection system can be configured to be easily attached to the visor with mechanical fasteners or other fastening means. Alternatively, the UVC disinfection system can be integrated into the visor and or headrest of an automobile.

To ensure safety of occupants (humans and/or pets), the UVC disinfection system can have one or more sensors (e.g., proximity sensor, motion sensor, infrared sensor, weight sensor, object recognition camera) configured to determine whether the interior of the vehicle is occupied. If the UVC disinfection system determines, based on sensor(s) readings, that the interior of the vehicle is empty, then the controller of the UVC disinfection system can turn on the UVC light to start the disinfecting process, which depending on the power of the UVC source, the volume and the complexity of the confined space to be disinfected, may last from minutes to hours.

The disinfection can be started manually by pushing a button giving the driver a pre-determined count-down time to step out of the car. When the disinfection is about to start, at count-down time zero, and the car is still occupied the disinfection is not started. By pushing a stop button the count-down is stopped and the disinfection is not started. In some embodiments the disinfection system has a small display to show the count-down time and the remaining disinfection time.

The UVC system can also be configured to be controllable via a mobile device using Bluetooth or other wireless communication technologies such as baseband (e.g., 5G). The UVC disinfection system can include a communication module with wireless communication capabilities such as, but not limited to, Bluetooth, near-field communication (NFC), broadband (Wi-Fi), and baseband communication (e.g., 4G, 5G).

In some embodiments, the controller of the UVC disinfection system with the ability for wireless communication such as Bluetooth or WIFI have remote sensors attached to the disinfection system, such as sensors to determine the occupancy of the car, connections to the CAN-bus of the car to determine car occupancy and/or whether the doors are locked, but also UVC measurement devices that can signal if the level of 50 mJ/cm² is reached, thus guaranteeing a disinfected volume rather than relying on disinfecting for a predetermined time.

During the UVC disinfecting process, the UVC disinfection system can continuously (or intermittently) monitor one or more sensors to determine whether there is any change in the status of a sensor. For example, if the door locks changed from a locked state to an unlocked state.

In some embodiments, a car key proximity sensor can be used to determine whether the driver is near-by and is about to unlock or enter the car. In this way, once the car key proximity sensor determines that the car key is nearby or approaching, the controller can disable the UVC light. Alternatively, a smartphone with dedicated app can be used, using the wireless connection to the disinfection system, to start, stop and monitor the disinfection cycle.

In some embodiments, the wireless connections of multiple disinfection system, can be used to synchronize the disinfection among the disinfection systems, e.g. the disinfection system at the visor of the driver seat is the master that controls the slave systems of the disinfection systems in the passenger seats, such as at the visor and/or headrests.

In some embodiments, the wireless connection of the disinfection system can be used to disinfect and monitor a fleet of cars in a garage.

The disinfection system may produce heath, which might excess a certain temperature level. Internal temperature sensors may throttle the power to the UVC sources to restrain the heath production, at which the remaining disinfection time is adapted to reach the required energy for disinfecting the volume.

As mentioned, the UVC disinfection system can be attached or integrated onto the visors of a vehicle. The UVC disinfection system can be equipped with one or more small motors to automatically swing and/or rotate the UVC light sources so that different areas of the vehicle can be treated. For example, rather than being fixed, the UVC light source can be rotatable about its longitudinal axis. This enables the UVC disinfection system to expose different areas of the passenger cabin to UVC light, typically in trucks or busses In additional to the UVC light source being rotatable, the UVC light source can be mounted on a support structure that can swing and/or rotate out at different angles. This further provides additional degrees of rotation and thereby enabling maximum surface coverage in a vehicle using a single UVC disinfecting unit.

### UVC Disinfection System

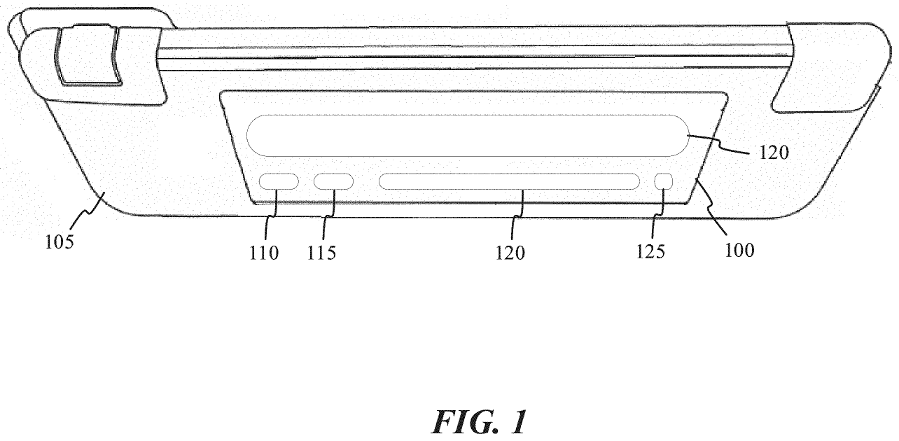

FIG. 1 illustrates a visor with a UVC disinfection system 100 in accordance with some embodiments of the present disclosure. UVC disinfection system 100 can be mounted onto visor 105 using mechanical fastener such as hooks-and-loops, straps, or screws. Alternatively, UVC disinfection system 100 can be integrated into the body of visor 105. UVC disinfection system 100 can also be mounted on various places in the interior cabin of a vehicle such as, but not limited to, the front and back pillars (support structures of the car roof and windshields), the ceiling of the interior cabin, on a seat headrest, trunk mount, back window, and interior roof rails.

UVC disinfection system 100 can include one or more sensors 110, 115 and one or more UVC light sources 120, which can be one or more strips of UVC light emitting diode (LED). The one or more light sources can also be mercury vapor lamps or fluorescent light bulbs, e.g. in case of alleys in busses and airplanes, where more irradiation power is needed.

Sensors 110, 115 can be a motion sensor, a proximity sensor, or an object recognition camera. UVC disinfection system 100 can also include other sensors placed at different places in the vehicle such as weight sensors, internal proximity sensors (for rear and front seats), external proximity sensors, and door lock sensors. In other words, one or more sensors (e.g., motion, proximity, UVC exposure sensor) can be remotely located from the main system components of UVC disinfection system 100. Each of the remotely located sensors can be in a wired or wireless communication with the communication module of the processor of the UVC disinfection system 100. The motion sensor can be an infrared (IR) sensor or other type of motion sensor such as an ultrasonic sensor or video camera. The proximity sensor can be photoelectric sensor, IR sensor, and capacitive sensor. The object recognition camera can include detection algorithms to trained to detect people and pets. One or more of these sensors can be used by UVC disinfection system 100 to determine whether the passenger space is occupied or empty. In this way, UVC disinfection system 100 would not accidentally enable the UVC light source while the passenger space is occupied.

In some embodiments, UVC disinfection system 100 includes door and door lock sensors. UVC disinfection system 100 is configured to turn on or off the UV light source (e.g., stop the cleaning process) when the sensor detects that the door is opened or the door lock goes from a locked to an unlocked position. UVC disinfection system 100 can also turn on or off the UVC light source based on the output of the interior and/or exterior proximity sensors.

One of the remote sensors of the UVC disinfection system 100 can include a biological fluid detection system (not shown), which can include a UV black light lamp 120, a camera 125, and biological fluid detection software module (not shown) configured to recognize biological fluid (e.g., saliva, blood) using images captured by camera 125 while the interior is exposed to the UV black light. Once a location is determined to have biological fluid or excess biological fluid, the controller (not shown) can swing and/or rotate the UV light source so that the location with detected biological fluid can be exposed to the UVC light. UVC disinfection system 100 can also include a reporting module (not shown) configured to report via wireless communication to the user's mobile device on treated areas, untreated areas due to the cleaning process being interrupted, areas detected with biological fluid, etc. In this way, the user can perform additional manual cleaning if necessary. For example, UVC disinfection system 100 can provide a map/image of the interior cabin of where the detected dirty (e.g., has biological fluid) areas are located. This option is especially viable for public transport and public toilet settings.

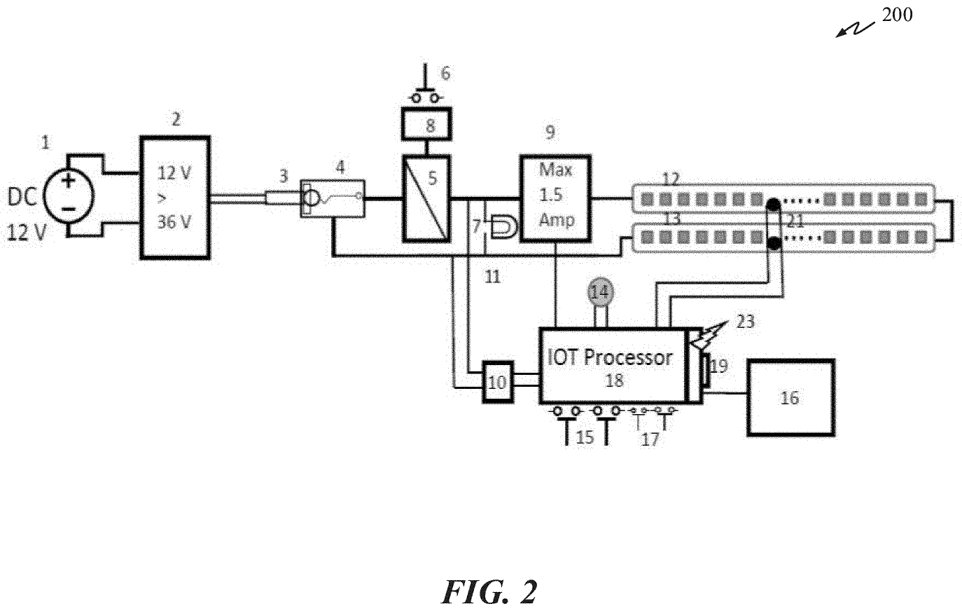

FIG. 2 illustrates a circuit diagram for a UVC disinfection system 200 in accordance with some embodiments of the present disclosure. UVC disinfection system 200 can include one or more components of UVC disinfection system 100, which can also be implemented with the components shown in FIG. 2. Similarly, UVC disinfection system 100 can include one or more components of UVC disinfection system 200 as described below. The circuit components of UVC disinfection system 200 are as follows: 1. 12 volt source (e.g., car battery, cigarette plug); 2. 12 Volt>36 Volt step up converter; 3. 36 Volt Plug; 4. 36 Volt Bus; 5. 36 Volt main relay; 6. Power on push button; 7. Power on LED; 8. Push button to on/off for relays; 9. Current limiter (Max 1.5 Amp) with PWM input to throttle the UVC LEDS; 10. 36 Volt>3.3 Volt step down converter for IOT processor; 11. Internal connector to split high voltage from low voltage PCBs; 12. 18 Volt 1.5 Amp UVC LED strip on heath sink in series with item 13; 13. 18 Volt 1.5 Amp UVC LED strip on heath sink in series with item 12; 14. IR Motion Detector; 15. Pushbuttons for start and stop; 16. OLED display; 17. Mini pushbuttons for set-up; 18. IOT processor; 19. mini-USB bus; 20. Visor shaped housing; 21. Heath sensor; 22. Velcro straps; 23. Blue Tooth/WIFI; and 24. Calibrated UVC-Sensor. Although not shown, circuit diagram 200 can include one or more components shown in FIG. 1 such as sensors 110 and 115, UV black light 120, and camera 125.

In some embodiments, current through the UV light source can be limited to 1.5 Amps in order to avoid excess heat. IR sensor 14 can be used to enable and disable the UV light source. For example, if IR sensor 14 detects heath and motion, then UV light source is disabled or the cleaning process stopped and/or will be postponed. UVC disinfection system 200 can also include a start delay button, in which the user can press to delay the cleaning cycle by a predetermined amount of time (e.g., 5, 10 minutes). UVC disinfection system 200 can also include an on/off button and/or an abort button.

Processor 18 can be configured to control the UV light source(s) based on outputs of one or more sensors such as IR sensor 14 and other sensors not shown on FIG. 2 (e.g., heat sensor, weight sensor, objection recognition camera). Display 16 can be used to display various information such as lead time, remaining cleaning time, settings menu, etc. Bluetooth/WIFI module 23 can be a wireless communication module with baseband communication capabilities. In this way, UVC disinfection system 200 can communicate with the user's mobile phone via Bluetooth or via the internet using a baseband communication system such as 4G or 5G.

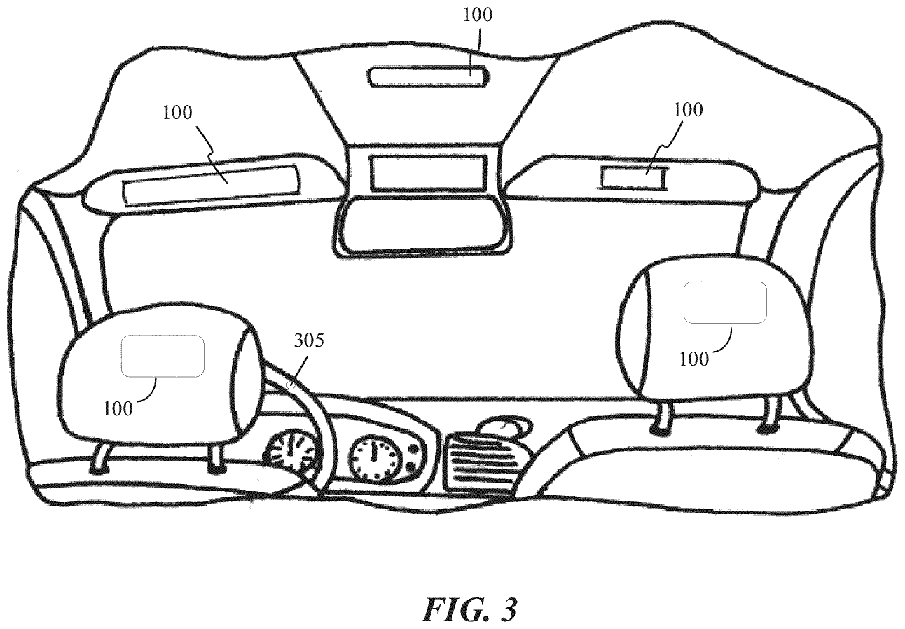

FIG. 3 illustrates an interior of a vehicle having one or more UVC disinfection systems 100 installed at various locations in accordance with some embodiments of the present disclosure. As shown, UVC disinfection system 100 (or 200) can be installed on the visors, ceiling of the passenger cabin, and back of headrests. Where multiple UVC disinfection systems are used, each system can be linked to one another so that information such as, but not limited to, cleaning schedule, cleaning areas, cleaning time, identified dirty areas, and sensor data can be shared. FIG. 3 also shows a UVC sensor 305 on the steering wheel. Although only one UVC sensor 305 is shown, the passenger cabin can have many sensors 305. Each UVC sensor 305 can be placed at strategic places (e.g., the steering wheel, radio control buttons) in the cabin where proper UVC exposure are necessary. UVC sensor 305 can also be configured to provide a feedback loop such that the controller can enable UVC light until each UVC sensor in the car has sensed enough UVC energy to sufficiently kill off any bacteria and/or viruses. This feedback safes energy, disinfection time and the effect of the (limited) photo-bleaching of surfaces. In addition the amount of energy for each sensor can be read out by a wirelessly connected device such as a smartphone.

UVC disinfection systems 100 disinfects air and surfaces within a particular volume of a vehicle previously described as a seated person's confined personal space. All surfaces within hands reach when seated should be disinfected. When more than one system is present in the vehicle the systems should be synchronously operated. Sufficient suitable remote sensors should be coupled to the one or more of the disinfection systems to guarantee that the vehicle is empty, e.g. CAN-bus sensor or IR presence sensor. By way of example in a system with only a driver's seat disinfection system a present back seat-passengers should not be hit by the UVC light of the driver's seat disinfection system when the driver is not present. Note that for taxi-services the back seats of the vehicle can be separated from the front-seats of the vehicle by using a glass or plexi-glass separation.

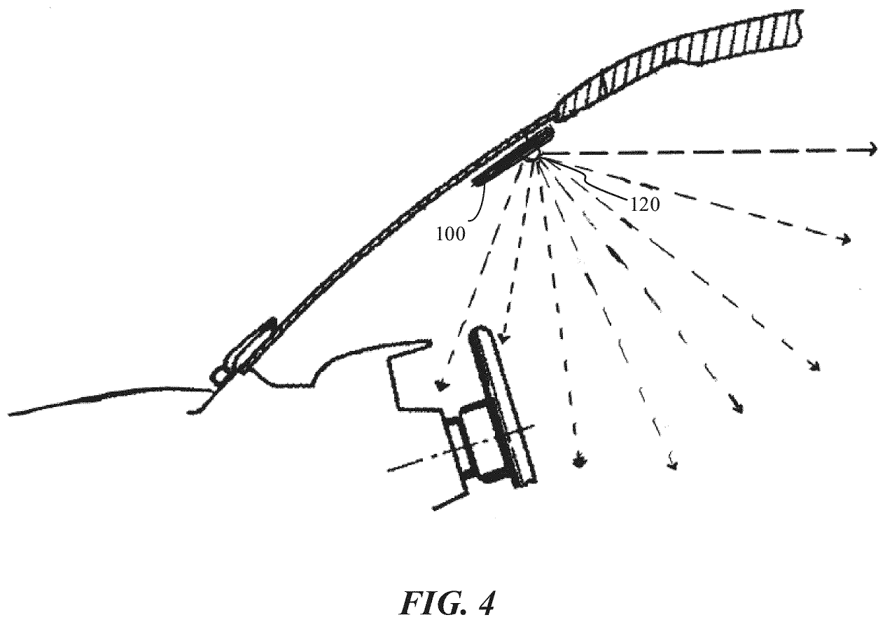

FIG. 4 illustrates a side view of an interior of a vehicle equipped with UVC disinfection system 100 having rotatable UVC light source 120 in accordance with some embodiments of the present disclosure. UVC light source 120 can have one or more strips of UVC LEDs. Generally, each UVC LED strip can expose a certain area simultaneously. For example, each strip can emit a beam of UVC light having an effective width of 5-30 degrees. This means that if the UVC strip is stationary, only certain areas of the interior cabin will be disinfected and some areas will remain untreated. Rotatable UVC light source 120 can solve this issue by rotating the UVC light to a different angle after a prerequisite cleaning time. For example, rotatable UVC light source 120 can first be rotated to point at and treat the steering wheel. After 10 minutes of exposure, rotatable UVC light source 120 can be rotated to direct the UVC light at the seat or headrest.

In some embodiments, UVC light source 120 can also be configured to rotate sideway—from the driver side to the passenger side. This can be done, for example, by mounting one or more UVC LED to a support structure that can rotate on two different axes that are perpendicular to each other. In this way, UVC light source 120 can be rotated from the steering wheel toward the rear of the cabin (back seat) and/or from the passenger side to the drive side. Working in conjunction with the biological fluid detector (i.e., UV black light 120 and camera 125), the controller (e.g., processor 18) can rotate UVC light source 120 to areas of the cabin with detected biological fluid.

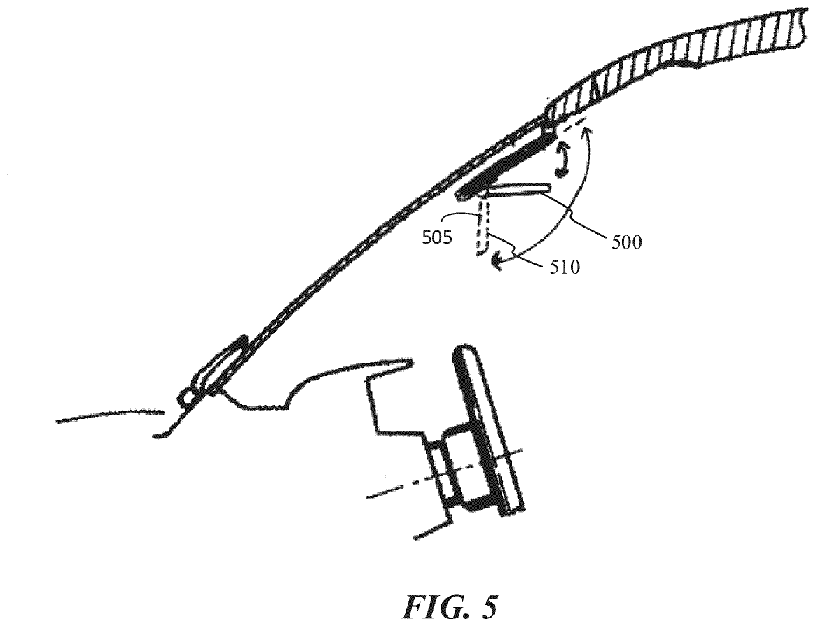

FIG. 5 illustrates a side view of an interior of a vehicle equipped with UVC disinfection system 100 being disposed on a hinged housing 500 that can be swing up and down in accordance with some embodiments of the present disclosure. Hinged housing 500 is configured to swing from a closed position to an open position, up to 180 degrees. Hinged housing 500 includes outer surface 505 and inner surface 510, each of which can have one or more UVC light sources 120. The rotation of hinged housing can also be controlled by the system's controller (e.g., processor 18). In this way, multiple areas such as the steering wheel and the headrest can be cleaned at the same time. In some embodiments, system 100 can be tilted by hand to ensure that the correct surfaces are hit by the UVC light. In another embodiment, this tilting can be achieved by mechanical means (motor) to extend the disinfection range, e.g., for driver cabins of trucks and busses.

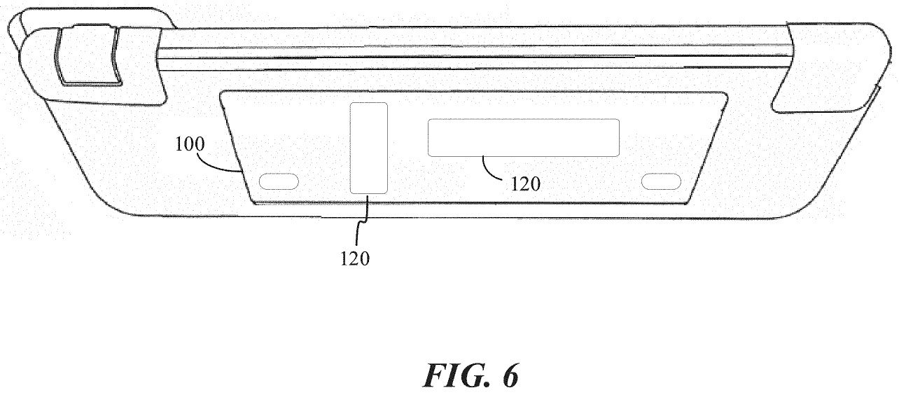

FIG. 6 illustrates UVC disinfection system 100 having UVC light sources oriented in different directions in accordance with some embodiments of the present disclosure. As shown, UVC disinfection system 100 can have multiple UVC light sources—one disposed horizontal with the visor/housing and one disposed perpendicular to the housing. Since each UVC light source can be rotated along its longitudinal axis, this arrangement can provide increased area of coverage and better control of exposure areas. In some embodiments, the visor can also be motorized such that the entire visor can swing from a closed position to an open position at any angle. This further increase the range of rotation and the areas that can be treated.

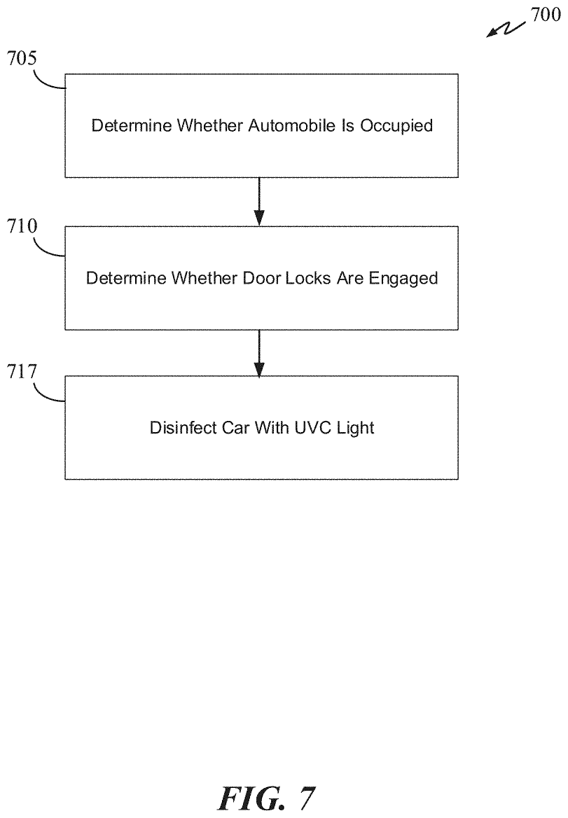

FIG. 7 illustrates a process 700 for disinfecting an interior of a vehicle in accordance with some embodiments of the present disclosure. Process 700 starts at 705 where one or more sensors (e.g., sensor 110, 115) are used to determine whether the passenger cabin is occupied or empty. For example, an IR sensor can be used to detect motion. If motion is detected, then the passenger cabin is occupied and the disinfection system would be disabled by the controller (e.g., processor 18). In another example, weight sensors below seats and/or proximity sensors disposed at various locations of the cabin can be used to determine if a person is sitting on a seat or is closed to a proximity sensor.

At 710, the locking status of locks of each door is determined. In some embodiments, if any lock is unlocked, then the disinfection system is disabled. At 717, if the passenger cabin (e.g., personal area/space) is empty and all doors are locked, then the disinfection system can start the disinfecting process. In some embodiments, 710 is optional. In this embodiment, the cleaning process can commence when it is determined that the passenger cabin is empty.

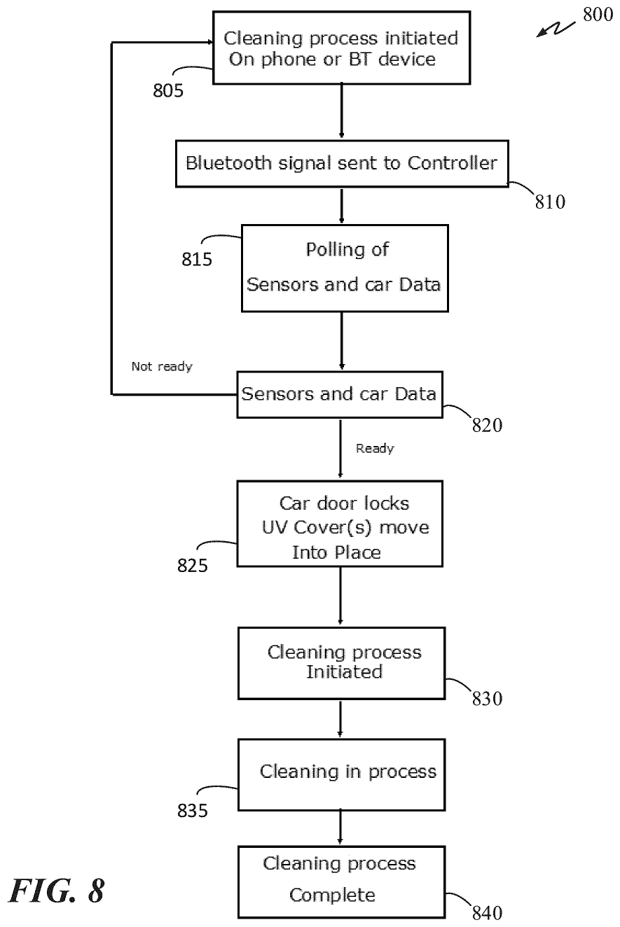

FIG. 8 illustrates a process 800 for disinfecting an interior of a vehicle in accordance with some embodiments of the present disclosure. Process 800 starts at 805 where the user can initiate the cleaning process from a mobile device, which can be communicatively connected to UVC disinfection system 100 via wireless communication (e.g., Bluetooth, 5G). At 810, the Bluetooth or wireless communication signal/data is sent to the controller of UVC disinfection system 100. At 815, UVC disinfection system 100 polls one or more sensors and vehicle data to determine whether the vehicle is occupied or empty. At 820, data from the one or more sensors are used to determine if the UVC disinfection system 100 can start (e.g., car is empty and doors are locked). If not ready, the system can continuously re-poll the sensors or can wait for the user to initiate the cleaning process again. At 825, UVC disinfection system 100 moves the UV light sources and/or the housing (e.g., rotatable support structure) into position. At 830, the cleaning process can start. In some embodiments, the UV light source can be rotated to clean different areas of the cabin. At 835, the cleaning process can rotate the UV light source to clean various areas. Additionally, UVC disinfection system 100 can use the biological fluid detection system to discover areas to clean and rotate and/or swing the housing and UV light source to clean the dirty areas accordingly. At 840, the system can automatically shut down when the cleaning process is completed.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the following detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

1. A disinfection system comprising:
a housing;
one or more UVC light sources configured to emit light having wavelength between 200 to 280 nm, wherein the one or more UVC light sources are mounted on the housing;
a sensor configured to determine whether an interior of a space is empty of occupants; and
a controller configured to control the one or more UVC lights based on the sensor determination of whether the interior of the space is empty.

2. The disinfection system of claim 1, wherein the sensor comprises a heat sensor.

3. The disinfection system of claim 1, wherein the sensor comprises an object recognition camera.

4. The disinfection system of claim 1, wherein the sensor comprises one or more proximity sensors.

5. The disinfection system of claim 1, wherein the housing is configured to be attached to a visor of a vehicle.

6. The disinfection system of claim 1, wherein the housing is integrated into a body of a visor of a vehicle.

7. The disinfection system of claim 1, wherein the one or more UVC light sources are rotatably mounted to the housing, wherein the controller is configured to rotate the one or more UVC light sources about an axis parallel to a main axis of the housing to disinfect different areas of the vehicle.

8. The disinfection system of claim 8, wherein the one or more UVC light sources are swingingly mounted to the housing, wherein the controller is configured to swing one or more ends of the one or more UVC light sources in a direction away from the housing.

9. The disinfection system of claim 1, wherein the housing is configured to be attachable to a roof of a vehicle.

10. The disinfection system of claim 10, wherein the one or more UVC light sources are rotatably mounted to the housing, wherein an axis of rotation is perpendicular to the housing.

11. The disinfection system of claim 1, wherein the interior of the space comprises an interior of a vehicle, and wherein the controller is configured to turn off the one or more UVC light sources upon detection that a door of the vehicle is open.

12. The disinfection system of claim 1, wherein the interior of the space comprises an interior of a vehicle, and wherein the controller is configured to turn off the one or more UVC light sources upon detection that a lock on a door of the vehicle is actuated from a locked to an opened position.

13. The vehicle disinfection system of claim 1, further comprising one or more UVC sensors disposed at one or more locations within the interior of the space, wherein the one or more locations are different from where the housing is located.

14. A vehicle visor comprising:
one or more UVC light sources configured to emit light having wavelength between 200 to 280 nm;
a sensor configured to determine whether the vehicle is empty of occupants; and
a controller configured to turn on the one or more UVC lights based on the sensor determination of whether the vehicle is empty.

15. The vehicle visor of claim 14, wherein the one or more UVC light sources are mounted on a housing such that the one or more UVC light sources can rotate about one or more axes.

16. A method for disinfecting a vehicle, the method comprising:
determining, using one or more sensors, whether the vehicle is empty of occupants and whether doors of the vehicle are locked; and
turning on a UVC light source to disinfect the vehicle based on whether the vehicle is empty of occupants and whether the doors are locked.

17. The method of claim 16, wherein determining whether the vehicle is empty of occupants comprises using a heat sensor to detect if there are occupants in the vehicle.

18. The method of claim 16, wherein determining whether the vehicle is empty of occupants comprises using an object recognition camera to detect if there are occupants in the vehicle.

19. The method of claim 16, wherein determining whether the vehicle is empty of occupants comprises using a proximity sensor to detect if there are occupants in the vehicle.

20. The method of claim 16, further comprising determining whether windows of the vehicle are fully closed, and wherein the UVC light source is turn on only when windows of the vehicle are fully closed.
